(19) United States
(12) Patent Application Publication
(10) Pub. No.: US 2013/0109620 A1
(43) Pub. Date: May 2, 2013

(54) BALAGLITAZONE COMPOSITIONS AND METHODS

(71) Applicants: Bente Riis, Morcote (CH); Kim Henriksen, Hillerod (DK); Rajinder Kumar, Cambridge (GB)

(72) Inventors: Bente Riis, Morcote (CH); Kim Henriksen, Hillerod (DK); Rajinder Kumar, Cambridge (GB)

(73) Assignees: RHEOSCIENCE A/S, Herlev (DK); REDDY'S LABORATORIES, LIMITED, Hyderabad (IN)

(21) Appl. No.: 13/516,917

(22) PCT Filed: Dec. 17, 2010
(86) PCT No.: PCT/IB2010/003287

§ 371 Date: Nov. 28, 2012

Related U.S. Application Data
(60) Provisional application No. 61/287,821, filed on Dec. 18, 2009.

(51) Int. Cl. A61K 31/517; A61K 45/06; A61K 38/28

(52) U.S. Cl. CPC A61K 31/517; A61K 38/28; A61K 45/06
USPC 514/6.5; 514/266.2; 514/171

(57) ABSTRACT

Methods for reducing bone loss in a subject suffering from a condition benefiting from a decrease in insulin resistance, by balaglitazone administration to the subject.

### INTRODUCTION

Aspects of the present application relate generally to balaglitazone, and methods for treating elevated insulin resistance by the administration of balaglitazone.

In aspects, the present application is directed to methods for decreasing insulin resistance without significant bone loss in subjects suffering from a condition benefiting from a decrease in insulin resistance, embodiments comprising administering balaglitazone to the subjects.

In aspects, the application is directed to methods for decreasing insulin resistance while concomitantly reducing the risks of bone fracture, embodiments comprising administering balaglitazone.

In aspects, the application is directed to methods for decreasing insulin resistance while concomitantly improving or maintaining bone density and/or bone mineral content, embodiments comprising administering balaglitazone.

In aspects, the application is directed to methods for decreasing insulin resistance while concomitantly decreasing or avoiding increases in the rate of bone degradation, embodiments comprising administering balaglitazone.

In aspects, the application is directed to methods for improving blood glucose control in subjects suffering from a condition benefiting from a decrease in insulin resistance, embodiments comprising administering balaglitazone to the subjects, wherein a ratio of efficacy to safety of balaglitazone is improved, as compared to the ratio of efficacy to safety for other thiazolidinedione drugs.

### BRIEF DESCRIPTION OF THE DRAWINGS

FIG. 1 shows baseline demographic and clinical data for subjects enrolling in a clinical study of the example.

FIG. 2 shows clinical data for subjects completing a clinical study of the example.

FIG. 3 shows biochemical parameters for subjects completing a clinical study of the example.

FIG. 4 shows adverse events experienced by subjects in a clinical study of the example.

### DETAILED DESCRIPTION

Reference will be made in detail to various embodiments of the application, an example of which is provided hereinbelow. The example is provided by way of explanation of the application, not a limitation of the application. In fact, it will be apparent to those skilled in the art that various modifications and variations can be made in the present application without departing from the scope or spirit of the application. For instance, features illustrated or described as part of one embodiment can be used in another embodiment, to yield a still further embodiment.

Thus, it is intended that the present application covers such modifications and variations as come within the scope of the appended claims and their equivalents. Other objects, features and aspects of the present application are disclosed in or are apparent from the following detailed description. It will be understood by persons having ordinary skill in the art that the present discussion is a description of exemplary embodiments only, and is not intended as limiting the broader aspects of the present application.

In the discussion below, citations to certain of the documents present in a list following the example are indicated by the numerals in parentheses.

Type 2 diabetes (“T2D”) is a major cause of morbidity and mortality in the industrialized world. The number of T2D patients is increasing rapidly, and is expected to reach between 300 and 380 million by 2025 (2, 3), thereby placing an enormous economic burden on global healthcare.

T2D is characterized by insulin resistance, hyperglycemia, and a loss of gamma-cell function, all of which lead to disruption of carbohydrate/lipid metabolism, and potentially result in deterioration of the microvasculature, leading to blindness and renal failure as well as several other complications (4). Clinical trials and meta-analyses have indicated that intensive glycemic control is important when treating T2D, as it reduces T2D deaths (5, 6).

Peroxisome proliferator-activator receptor-gamma (“PPAR-γ”) is a ubiquitously expressed receptor, which, upon activation, elicits gene transcription of a large number of genes (4, 7). Activation of PPAR-γleads to a plethora of effects in various tissues, including liver, fat, kidney, muscle, and bone (4).

Thiazolidinediones (“TZDs”) are PPAR-γ agonists that are widely used to treat T2D due to their powerful ability to control glucose levels (4). However, use of TZDs is also associated with significant adverse events, of which heart failure (5, 6, 8, 9), weight gain due to accelerated adipogenesis (10, 11), peripheral oedemas (12, 13), and bone loss due to direct inhibition of bone formation by osteoblasts (14-19) are included.

PPAR-γ agonists require either full or partial activation of PPAR-γ to mediate their function (20, 21). Glycemic improvements may be achieved by partial agonists, while reducing some of the unwanted side effects elicited by full agonist activation (22-24). This indicates that, in vivo, the optimal balance between optimal glycemic control and minimum detrimental effects may lie in partial PPAR-γ agonists.

Balaglitazone, having a chemical name 5[4-(3-methyl-4-oxo-3,4-dihydroquinazolin-2-ylmethoxy)-benzyl]-thiazolidine-2,4-dione, is a PPAR-γ agonist. Balaglitazone elicits about 52% of the PPAR-γactivation observed with the full agonist rosiglitazone. The structure of balaglitazone is shown below.

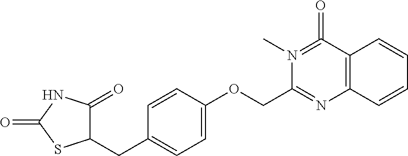

Aspects of the present application are directed to methods for reducing bone loss in subjects being treated for elevated insulin resistance, by administering to the subjects an effective amount of balaglitazone or a salt thereof, such as the potassium salt. A reduction in bone loss may be established, for example, by comparing net bone loss over a time period in a first group of subjects, to whom balaglitazone is administered at a dosage of 10 mg per day, with that of a second group of subjects to whom balaglitazone is administered at a dosage of 20 mg per day and a third group of subjects to whom pioglitazone is administered at a dosage of 45 mg per day, the subject groups being substantially equivalent in terms of at least age, sex, weight, and severity of condition. In certain embodiments, the reduction in bone loss may be observable when the third group of subjects is administered 30 mg pioglitazone per day. In embodiments, the subject groups comprise at least 50 subjects. In embodiments, the subject groups comprise at least 100 subjects.

If an individual belongs to a demographic group similar to those of the first or second subject groups, the bone loss may be reduced upon balaglitazone administration. It is not required that the rate of bone loss in such an individual decreases upon treatment with balaglitazone in comparison to their own rate of bone loss prior to the treatment. Bone loss may be determined by measuring bone density or bone mineral content (optionally on a total body basis, but alternatively at a specific site, e.g., the spine or hip) at the beginning and ending of the treatment period and determining a difference between those measurements.

Some of the bone health benefits encompassed by embodiments of the present application include one or more of: a reduction in bone loss or fracture; a reduction in risk of bone loss or fracture; the increase or maintenance of bone mineral content and/or bone mineral density; the decrease or avoidance of an increase in bone degradation; a decrease in risk for osteoporosis; a decrease in the formation of osteoclasts; an inducement of apoptosis of osteoclasts; an increase in bone formation via an increase in alkaline phosphatase; maintenance of alkaline phosphatase levels; maintenance of serum osteocalcin levels; an increase in calcium absorption; an increase in bone calcium content; an increase in bone-DNA content; an increase in hormones involved in bone formation, such as osteoprotogerin, osteopontin, osteocalcin, or insulin-like growth factor-1 (“IGF-1”); an increase in collagen; an increase in transcription factors involved in bone formation, such as runt-related transcription factor 2 (“RUNX2”); and increase of osteoblasts.

In embodiments, the serum bone alkaline phosphatase level of a subject after balaglitazone administration is substantially maintained at a pre-administration level. In embodiments, the serum osteocalcin level of a subject after balaglitazone administration is substantially maintained at a pre-administration level. In embodiments, the bone mineral density of a subject after balaglitazone administration is substantially maintained at a pre-administration level.

In embodiments, a subject to be treated has a condition including one or more of osteoporosis, achondroplasia, bone and joint infections, bone cancer, bone fractures, craniosynostosis, curvature of the spine, enchondroma, fibrodysplasia ossificans progressive, fibrous dysplasia, giant cell tumor of bone, infectious arthritis, Klippel-feil syndrome, Legg Calve Perthes disease, myeloma, Osgood-Schlater disease, Osteitis Condensans IIii, osteochondritis dissecans-OCD, osteochondroma—bone tumor, osteogenesis, osteomalacia, osteomyelitis, osteonecrosis, osteopenia, osteopetrosis, osteophytes, Paget's disease, Proteus syndrome, renal osteodystrophy, rickets, scoliosis, charcot joint, diabetic hand syndrome, osteoarthritis, diffuse idiopathic skeletal hyperostosis, Dupuytren contracture, frozen shoulder, and unicameral bone cyst.

In embodiments, a subject is evaluated by conducting at least one measurement of bone density, bone mineral content, and rate of bone degradation, and establishing that the subject has an increased risk of bone fracture.

In embodiments, the subject is a human adult. The adult subject may, in some embodiments, have a body mass index of at least 25 kg/m². In some embodiments, the subject may have a blood glycosylated hemoglobin (“HbA_{1c}”) level of at least 7%.

In embodiments, balaglitazone may be administered to subjects in amounts ranging from about 1 mg to about 100 mg per day. In embodiments, balaglitazone may be administered to subjects in amounts ranging from about 1 mg to about 25 mg per day. In embodiments, balaglitazone may be administered to subjects in amounts ranging from about 7 mg to about 15 mg per day. In embodiments, balaglitazone may be administered to subjects in amounts ranging from about 9 mg to about 12 mg per day. In embodiments, balaglitazone may be administered to subjects in amounts ranging from about 9.5 mg to about 10.5 mg per day. In particular embodiments, balaglitazone may be administered to subjects in an amount about 10 mg per day. In another particular embodiment, balaglitazone may be administered to subjects in an amount about 5 mg per day. The dosage levels for administration are not limited by the above ranges and may be determined based upon the typical or the best practices of those skilled in the art, as they exist now or may exist in the future.

These amounts of balaglitazone can be administered in a single daily dose, or can be administered in divided doses, such as twice daily or three times daily.

In embodiments, balaglitazone is administered for at least 52 weeks, or for at least 26 weeks, or for at least 13 weeks. In embodiments, balaglitazone is administered for at least 2 years or at least 3 years. In embodiments, balaglitazone is administered for at least 5 years.

In embodiments, this application comprises administering balaglitazone in combination with a therapeutic agent for restricting bone loss. The therapeutic agents include. without limitation thereto; one or more of vitamin A, vitamin D2, vitamin D, calcium, analgesics, non-steroidal anti-inflammatory drugs, cyclooxygenase-2 inhibitors, acetaminophen, chemotherapy drugs, antibiotics, antifungals, corticosteroids such as prednisone, and cinacalcet hydrochloride.

In embodiments, the application is directed to the use of balaglitazone in the manufacture of a medicament for the treatment of elevated insulin resistance, wherein the medicament additionally provides one or more of the bone health benefits discussed above.

In embodiments, the application comprises administering balaglitazone in combination with other anti-diabetic drugs. The anti-diabetic drugs include, but are not limited to, insulin, including derivatives and analogues thereof, insulin secretagogues (also called insulin secretion enhancers and insulinotropic agents), insulin sensitizers, biguanides, α-glucosidase inhibitors, potassium channel openers, glucagon antagonists, protein tyrosine phosphatase inhibitors, glucokinase activators, RXR agonists, hormone sensitive lipase inhibitors, glycogen synthase kinase-3 inhibitors, glycogen phosphorylase inhibitors, glucose uptake modulators, and lipid lowering compounds.

Useful insulin and derivatives and analogues thereof include human insulin and derivatives and analogues thereof. The term “human insulin” as used herein refers to naturally produced insulin or recombinantly-produced insulin. Recombinant human insulin may be produced in any suitable host cells. For example, the host cells may be bacterial, fungal (including yeast), insect, animal, or plant cells. The expression “insulin derivative” as used herein refers to human insulin or an analogue thereof, in which at least one organic substituent is bound to one or more of the amino acids. By “analogue of human insulin” as used herein (and related expressions) is meant human insulin in which one or more amino acids have been deleted and/or replaced by other amino acids, including non-codeable amino acids, or human insulin comprising additional amino acids, i.e., having more than 51 amino acids, wherein the resulting analogue possesses insulin activity in a subject.

Useful insulin secretagogues include sulfonylureas, meglitinides, and dipeptidyl peptidase (DPP) inhibitors. Useful sulfonylureas include tolbutamide, glibenclamide, gliclazide, glimepiride, glipizid, chlorpropamide, tolazamide, and glyburide. Useful meglitinides include nateglinide and repaglinide. Useful DPP inhibitors include DPP-IV inhibitors, such as sitagliptin, vildagliptin, saxagliptin, linagliptin, and alogliptin. Useful insulin sensitizers include troglitazone, ciglitazone, pioglitazone, rosiglitazone, isaglitazone, darglitazone, or englitazone.

Useful biguanides include metformin. Useful glucosidase inhibitors include voglobose, emiglitate, miglitol, and acarbose. Useful potassium channel openers include diazoxide. Useful lipid lowering compounds include statins, fibrates, and PPAR-γ agonists. Useful statins include atorvastatin, lovastatin, pravastatin, simvastatin, fluvastatin, and cerivastatin. Useful fibrates include fenofibrate, gemfibrozil, bezafibrate, and any other PPAR agonist.

As appropriate, balaglitazone and the other anti-diabetic compounds may be used in the form of the free acids or bases, rather than as a salt, or as a pharmaceutically acceptable salt rather than as free acids or bases. The use of prodrugs or solvates of the other anti-diabetic compounds is also contemplated in the present application. The term “prodrug” is intended to indicate a compound which does not necessarily have a therapeutic activity, but which upon administration is transformed in the body to the therapeutically active compound. Often, this transformation relies on enzymatic activity in the body, or on acid-base catalyzed reactions in the intestines.

In embodiments, a subject will receiving insulin therapy in addition to balaglitazone administration. In embodiments, a subject is receiving stable insulin therapy in addition to balaglitazone administration. The insulin therapy may, in embodiments, include treatment with insulin at doses at least 30 IU/day (±4 IU/day). In embodiments, the insulin therapy may include treatment with insulin at doses between about 10 IU/day and about 400 IU/day.

In embodiments of the application, the administration of balaglitazone results in improved blood glucose control. The subjects experiencing improved blood glucose control from balaglitazone administration may also have an increased risk of bone fracture for their age, sex, and weight demographics. The application further comprises the administration of balaglitazone for use in the treatment of a condition benefiting from a decrease in insulin resistance so as to bring about a reduction in bone loss or a reduction in the risk or extent of bone fracture, or decreasing or avoiding an increase in the rate of bone degradation in treated subjects.

In embodiments, a subject is suffering from a condition which would benefit from a decrease in insulin resistance. In such embodiments, the condition benefiting from a decrease in insulin resistance may include one or more of type 2 diabetes, dyslipidemia, hyperglycemia, hyperinsulinemia, insulin resistance, obesity, cardiovascular complications, atherosclerosis, hypertension, impaired glucose tolerance, impaired fasting glucose level, increased plasma levels of free fatty acids, increased plasma levels of triglycerides, and increased plasma levels of very low density lipoproteins (“VLDL”).

In some embodiments, balaglitazone administration may lower HbA_{1c} levels in a subject. In other embodiments, balaglitazone administration may lower fasting serum glucose levels in a subject. In still other embodiments, balaglitazone administration may lower post-prandial glucose levels in a subject. In some embodiments, balaglitazone administration may increase the probability of reducing insulin doses and/or decrease the probability of increasing insulin doses for a subject.

In various embodiments, balaglitazone administration may limit or reduce fat accumulation in a subject. For example, administration of balaglitazone may limit the weight gain of a subject, when compared to treatment with other thiazolidinedione drugs. Similarly, balaglitazone administration may maintain the lower leg weight of subjects during administration. Further, balaglitazone administration may increase or maintain lean tissue mass development, and/or decrease or limit fatty tissue mass development, in a subject.

In embodiments, balaglitazone administration may improve the lipid profile of a subject. Such improvement may include increasing high density lipoprotein (“HDL”) cholesterol levels, maintaining low density lipoprotein (“LDL”) cholesterol levels, and/or maintaining total cholesterol levels in a subject.

In embodiments, balaglitazone administration may reduce fluid retention in a subject. In embodiments, balaglitazone administration may reduce systolic and/or diastolic blood pressures in a subject.

In embodiments, the risk of heart failure, peripheral oedema, and myocardial infarction may be reduced by balaglitazone administration to a subject. Compared to other thiazolidinedione drugs, balaglitazone may produce fewer adverse effects.

In embodiments, the ratios of efficacy to safety of balaglitazone are improved, as compared to the ratios of efficacy to safety for other thiazolidinedione drugs, such as rosaglitazone, pioglitazone, and/or troglitazone. In embodiments, the ratios of efficacy to safety of balaglitazone are improved, as compared to the ratios of efficacy to safety for pioglitazone. More specifically, the ratios of efficacy to safety of balaglitazone are improved, as compared to the ratios of efficacy to safety for a 45 mg or 30 mg dose of pioglitazone, administered daily.

Balaglitazone for use according to the present application may be administered alone or in combination with pharmaceutically acceptable carriers or excipients, in either single or multiple doses. Pharmaceutical compositions may be formulated with pharmaceutically acceptable carriers or diluents, as well as any other known adjuvants and excipients, in accordance with conventional techniques.

In embodiments, balaglitazone may be provided in a unit dosage form containing a convenient amount for use in desired dosage regimes. In embodiments, the unit dose of balaglitazone may be 2.5 mg, 5 mg, 10 mg, or 25 mg, each ±10%.

Administration of balaglitazone may occur from one to four times daily. In a particular embodiment, administration occurs promptly following breakfast.

The pharmaceutical compositions may be specifically formulated for administration by any suitable route, such as oral, rectal, nasal, pulmonary, topical (including buccal and sublingual), transdermal, intracisternal, intraperitoneal, vaginal, and parenteral (including subcutaneous, intramuscular, intrathecal, intravenous and intradermal) routes.

Pharmaceutical compositions suitable for oral administration include solid dosage forms, such as hard or soft capsules, tablets, troches, dragees, pills, lozenges, powders, and granules. Where desired, they can be prepared with coatings, such as enteric coatings, or they can be formulated to provide controlled release of the active ingredients, such as delayed, sustained, or delayed-sustained release, according to methods known in the art.

Liquid dosage forms suitable for oral administration include solutions, emulsions, aqueous or oily suspensions, syrups and elixirs. Pharmaceutical compositions for parenteral administration may include sterile aqueous and non-aqueous injectable solutions, dispersions, suspensions, or emulsions, as well as sterile powders to be reconstituted in sterile injectable solutions or dispersions prior to use. Depot injectable formulations are also contemplated as being within the scope of the present application.

Other suitable administration forms include suppositories, sprays, ointments, creams, gels, inhalants, dermal patches, and implants.

For topical use, creams, ointments, gels, solutions, and/or suspensions containing the compounds of the present application are contemplated. For the purpose of this application, topical applications include mouth washes and gargles.

Compositions intended for oral use may be prepared according to any methods, and such compositions may contain one or more agents such as sweetening agents, flavoring agents, coloring agents, and preserving agents, in Order to provide pharmaceutically elegant and palatable preparations.

The compounds for use according to the present application may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

If a solid carrier is used for oral administration, the preparation may be tableted or placed into a hard gelatin capsule in powder or pellet form, or it can be in the form of a troche or lozenge. The amount of a solid carrier will vary widely, but will frequently be from about 25 mg to about 1 g. If a liquid carrier is used, the preparation may be in the form of a syrup, emulsion, soft gelatin capsule, or sterile injectable liquid such as an aqueous or non-aqueous liquid suspension or solution.

Subjects to be treated according to embodiments of the application are mammals, including human subjects of either sex.

The following example describes various embodiments of the present application. Other embodiments within the scope of the claims herein will be apparent to one skilled in the art from consideration of the specification as disclosed herein. It is intended that the specification, including the example, be considered to be exemplary only.

A randomized, double-blind, parallel-group, placebo- and active comparator-controlled study was conducted to determine the efficacy and safety of balaglitazone in T2D patients on stable insulin therapy, with an emphasis on fat accumulation, fluid retention, and bone safety aspects. The study was conducted in Denmark, Sweden, and Finland and involved a 26-week treatment period comparing the efficacy and safety of two doses of balaglitazone (10 mg and 20 mg), placebo, and 45 mg pioglitazone (ACTOS® tablets) once daily and, after the last treatment, a 4-week follow-up visit to assess safety.

ACTOS tablets, sold by Takeda in strengths of 15, 30, and 45 mg of pioglitazone, contain, in addition to the drug (pioglitazone hydrochloride), lactose monohydrate, hydroxypropylcellulose, carboxymethylcellulose calcium, and magnesium stearate. The contained drug has a chemical name [(±)-5-[[4-[2-(5-ethyl-2-pyridinyl)ethoxy]phenyl]methyl]-2,4-]thiazolidinedione monohydrochloride,

Subjects were required to meet the following criteria to be included in the study: type 2 diabetes mellitus being diagnosed at least three months earlier according to the 1999 World Health Organisation criteria; age ≧18 years; body mass index (“BMI”)≧25 kg/m²; HbA_{1c}≧7%; and treatment with insulin on a stable dose of at least 30 U/day (±4 U/day), for at least 75 days, although insulin adjustments to resolve short term acute disease were permitted.

Exclusion criteria included the following: prior or current use of a PPAR-γ agonist; hospitalization for a major cardiovascular event in the previous 3 months; a scheduled major cardiovascular intervention; diagnosed or receiving medication for heart failure (stages I to IV of the New York Heart Association classification system); uncontrolled treated or untreated systolic blood pressure >180 mmHg and/or diastolic blood pressure >95 mmHg; serum creatinine >130 μmol/L; ALT, AST, total bilirubin or alkaline phosphatase 2.5 times the upper limit of normal; hemoglobin significantly, in the investigator's opinion, but not more than 1 mmol/L, below the lower limit of normal or hemoglobinopathy interfering with a valid HbA_{1c} assay; hematuria, defined as any, even a trace of, hematuria on a urinary dipstick at the screening or randomisation visit; known diabetic macular oedema; contraindication to or intolerance of study medication; a pre-existing medical condition judged to preclude safe participation in the study; abuse of alcohol or drugs, or presence of any condition that in the investigator's opinion might have led to poor adherence to the study protocol; recent use (<3 months) of an investigational drug; use of any drug, such as systemic corticosteroids, which in the investigator's opinion could interfere with the glucose level; diagnosis of clinically significant disease/disorder which in the investigator's opinion could interfere with the results of the trial; and planned surgery. Also excluded were women who were pregnant, breast feeding, planning a pregnancy, or not using adequate contraceptive methods, defined as an intrauterine device or oral contraceptives.

### PROCEDURE

Eligible subjects were instructed to discontinue all oral anti-diabetic drugs. Subjects were randomly assigned to double-blind treatment with 10 mg of balaglitazone, 20 mg of balaglitazone, 45 mg of pioglitazone (ACTOS®), or placebo. Randomization was performed by an appointed contract service provider, using blocks of four subjects per block with four treatment labels (placebo, balaglitazone 10 mg, balaglitazone 20 mg, and pioglitazone 45 mg). The patient to be randomized was always assigned the lowest randomisation number available at the respective investigator site.

Each study drug was given as a single tablet, once daily at breakfast, for the duration of the 26-week treatment period. All tablets were contained in an identical capsule. Subjects, study-site personnel, and sponsors were all blinded.

One screening visit was conducted four weeks before randomization, and at week zero, subjects meeting the inclusion and exclusion criteria were assessed for safety. Subjects visited the clinics at the screening visit, at baseline, and at weeks 4, 8, 12, 16, 20 and 26. At each visit, fasting blood samples were collected for analysis of glycemic control, lipids, and clinical chemistry. All laboratory analyses were performed by a central laboratory.

Subjects were instructed to keep their usual diet. They were supplied with a plasma glucose meter and instructed regarding how to obtain 7-point plasma glucose profiles. The values of the 7-point measurement performed within the last week before visits 2, 3, 4, 5, 6, 7 and 8 were included in the study. The 7-point plasma glucose profile consisted of glucose levels before each meal (breakfast, lunch, and dinner), 90 minutes after the start of each of those meals, and at bedtime.

Lower leg volume of both legs was determined using the volume of water displacement (“VWD”) method at visits 2 and 8, using Partial Leg Baseline Volumetric Edema Gauges (26).

Body composition was measured by Dual Energy X-ray Absorptiometry (“DXA”) on visits 2 and 8, in which total body lean (“LTM”), fat tissue mass (“FTM”), and bone mineral density (“BMD”) were measured. Subjects were instructed to fast, beginning at 10 PM the night before body composition measurements. To standardize the measurements of DXA and to avoid prolonged fasting, the patient had a standardized meal replacement after collection of fasting blood samples and just before scanning. The scans were conducted using Lunar DPX (GE Healthcare Worldwide), Hologic™ (Hologic™, Hologic (UK) Limited, UK), or Norland (Norland Medical Systems, Inc.) whole body scanners, and each individual was scanned with the same scanner at both visits.

At visits 1 and 8, physical examinations were conducted on subjects' ears, eyes, nose, throat, neck, respiratory system, cardiovascular system, and gastrointestinal system, with specific checks for peripheral oedema and cardiovascular events. Vital signs (diastolic and systolic blood pressure and pulse) were recorded after resting 5 minutes in a supine position at every clinic visit. A standard 12-lead ECG was performed at visits 1 and 8 and evaluated by the investigator. Any abnormalities were registered.

### STATISTICAL ANALYSIS

A sample size of 100 subjects per treatment group was chosen to ensure 80% power to detect a treatment difference of at least −1% (balaglitazone versus placebo) for HbA_{1c} change from baseline to the end of the treatment period, assuming a common standard deviation of 1.5%, at a one-sided significance level of 0.025 (primary endpoint). The sample size also ensured 80% power to detect a treatment difference of at least −1.5 kg (balaglitazone versus 45 mg pioglitazone) for body weight change from baseline to the end of the treatment period, assuming a common standard deviation of 3.5 kg, at a one-sided significance level of 0.025 (secondary endpoint). The primary endpoint was assessed with analysis of covariance (“ANCOVA”) to determine least squares (“LS”) means of change from baseline. The ANCOVA model included treatment and country as fixed effects and baseline HbA_{1c} as a covariate. A pair-wise comparison of each balaglitazone dose regimen against placebo was performed with a hierarchical decision procedure starting with the highest dose of balaglitazone at a two-sided significance level of 0.05. Comparisons between each balaglitazone dose regimen and pioglitazone and between pioglitazone and placebo were descriptive in nature, and significance levels were adjusted for multiple comparisons by the Tukey Kramer's method. Similar ANCOVA models were used to calculate least square means of change or percentage change from baseline and confidence intervals for secondary efficacy endpoints. For these secondary endpoints, the hierarchical decision procedure was not incorporated into the analysis. Fishers' exact test was applied for comparison of the frequencies of the adverse events of special interest: heart failure, ischemic heart disease, and peripheral oedema among all four groups, but also between the three treatment groups only. All subjects who received at least one dose of randomized study drug and who had an evaluable baseline and at least one evaluable post-baseline efficacy measurement were included in the efficacy analysis. All subjects who received at least one dose of randomized study drug were included in the safety analysis.

### Results

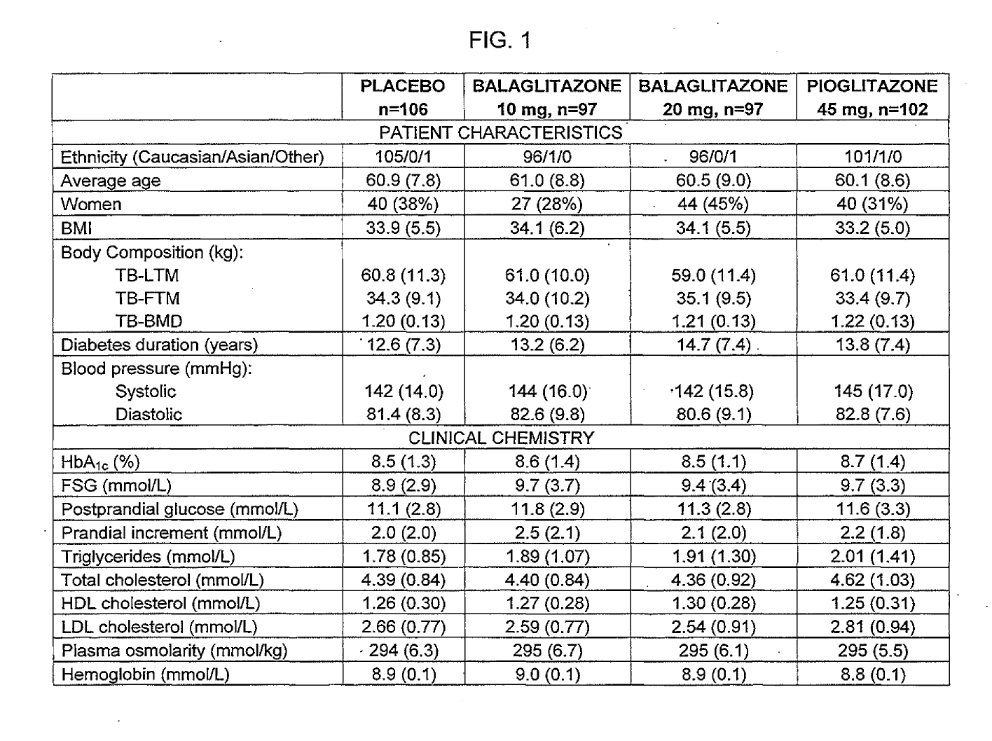

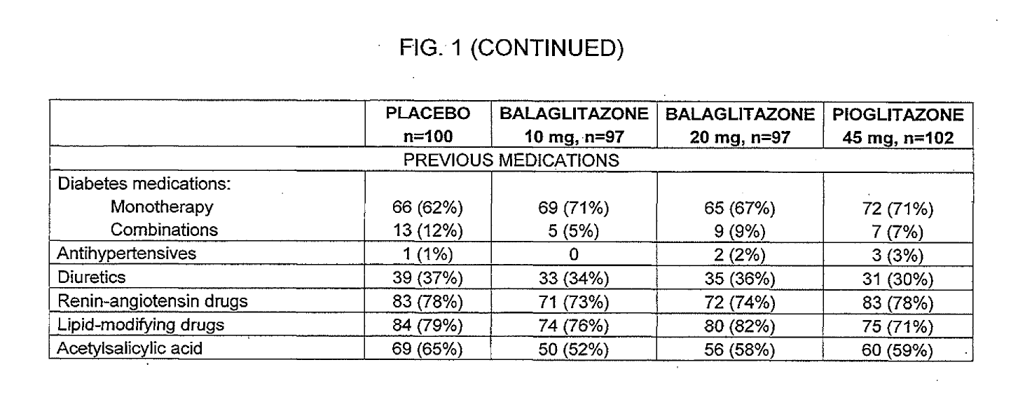

In total, 409 subjects (271 from Denmark, 99 from Finland, and 39 from Sweden) were enrolled in the treatment part of the trial, and all were randomized into the four study groups illustrated in FIG. 1, where n is the sample size and where table values in parentheses are standard deviations (except for the Previous Medications, where percentages of subjects are in parentheses). The efficacy population excluded 7 subjects (3 from placebo, and 2 from each of the two balaglitazone groups), and therefore included 402 subjects. Baseline demographic and clinical data, as well as previous medications, were all well-balanced between the four study groups, with the exception of gender, where men were over-represented in the study.

The mean duration of diabetes was 13.5 years. The majority of the study participants had been using oral anti-diabetic drugs before enrolling; however, use of anti-diabetic drugs except insulin was not allowed during the active part of the trial. In alignment with the criterion of poorly controlled diabetes, all four groups showed HbA_{1c} levels between 8.5 and 8.7%, and fasting serum glucose levels above 8.9 mmol/L, despite being on stable insulin therapy.

HbA_{1c}, fasting serum glucose (“FSG”), and mean post-prandial glucose levels were suppressed in all three treatment groups and, based on trends, the magnitude of suppression was the same in all groups. The placebo group, on the other hand, showed increased levels of all three parameters.

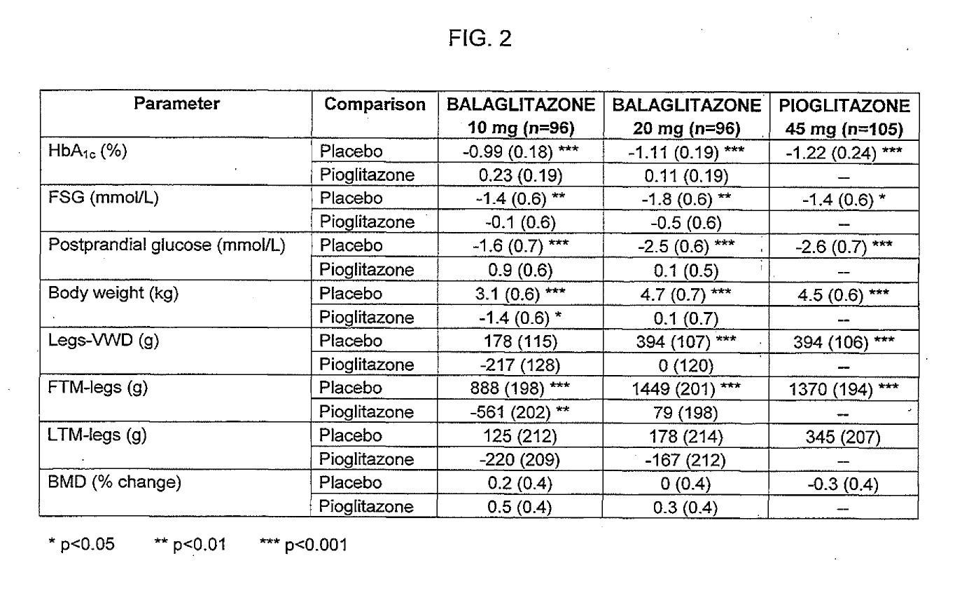

In both the 10 mg and 20 mg balaglitazone-treated groups, HbA_{1c} levels were significantly decreased by −0.99% and −1.11% respectively, as they were in the pioglitazone 45 mg group (−1.22%), when compared to placebo. All decreases were statistically significant (p<0.0001), as shown in FIG. 2, where values in parentheses are standard deviations.

Similar effects were observed with FSG, where reductions of −1.4 mmol/L and −1.8 mmol/L were observed in the balaglitazone 10 mg (p=0.01) and 20 mg (p<0.001) groups when compared to placebo. In the pioglitazone 45 mg group, a reduction of −1.4 mmol/L was observed when compared to placebo (p<0.05). With respect to post-prandial glucose levels measured using the 7-point glucose test, balaglitazone 10 mg, balaglitazone 20 mg and pioglitazone 45 mg led to significant reductions of −1.6 mmol/L (p<0.01), −2.5 mmol/L (p<0.0001), −2.6 mmol/L (p<0.0001) when compared to placebo. Furthermore, all three treatment groups reduced the serum insulin level by approximately 25%, when compared to placebo, although in the pioglitazone 45 mg group, only a trend was observed (p=0.10).

All three treatments lead to a significantly increased probability for reducing the insulin dose, as well as a significantly decreased probability for increasing the insulin dose. Finally, between 14% and 21% of subjects in the treatment groups reached the secondary endpoint of HbA_{1c}<7%, whereas only 2% of the placebo group achieved this.

With respect to hypoglycaemic episodes, 51% to 55% of participants in the treatment groups experienced these, while 30% of the placebo group experienced hypoglycaemia. No differences in the severity of the hypoglycaemic episodes, major and minor, were observed between the treatment groups.

Assessment of body weight in the balaglitazone 10 mg and 20 mg groups showed increases of 3.1 kg (p<0.0001) and 4.7 kg (p<0.0001), when compared to placebo. In the pioglitazone group, a weight increase of 4.5 kg (p<0.0001) was observed when compared to placebo. The balaglitazone 10 mg group showed a lower weight gain than the pioglitazone 45 mg group (p<0.02). BMIs in the four study groups showed the same significant changes as body weights.

Measurement of the volume of water displacement (“VWD”) showed no significant increase in leg volume in the balaglitazone 10 mg group, compared with placebo, although a trend toward increased volume was observed. In contrast, both the balaglitazone 20 mg and the pioglitazone 45 mg groups showed significantly increased lower leg volume, when compared with placebo.

Further analysis of the body composition using DXA scans showed a trend toward lower lean tissue mass (LTM) in the legs of the two balaglitazone groups compared to pioglitazone, although statistically non-significant. On the other hand, analysis of the fat tissue mass (FTM) in the legs confirmed a mean weight increase in all three treatment groups, with the balaglitazone 10 mg group gaining significantly less fat mass during the study than the pioglitazone group (p<0.01). Fat accumulation was also measured in the whole body, and all three treatments significantly increased whole body FTM compared to placebo. A trend toward lower fat accumulation was seen in the balaglitazone 10 mg group, compared to pioglitazone 45 mg.

Total body bone mineral density (“TB-BMD”) was analyzed based on the DXA scans and, although no significant changes were observed over the 26 weeks of treatment, pioglitazone displayed a trend toward loss of bone, compared with placebo. On the other hand, neither of the balaglitazone groups showed any indication of bone loss, when compared to placebo. A significant reduction in serum alkaline phosphatase (“ALP”) was observed in all three treatment groups compared with placebo. The reduction was significantly smaller in the balaglitazone 10 mg group than the pioglitazone 45 mg group.

With respect to lipids, treatment for 26 weeks with both doses of balaglitazone led to an improvement in the lipid profile when compared to placebo, as the HDL cholesterol levels were significantly increased (p<0.001 for both balaglitazone groups), while neither LDL cholesterol levels nor total cholesterol changed as a function of treatment. A similar pattern was obtained for pioglitazone 45 mg.

Measurements of systolic and diastolic blood pressures showed that balaglitazone treatment led to reductions in systolic (10 mg-1.5 mmHg (standard error (“SE”) 0.9) and 20 mg-4.5 mmHg (SE 1.9) respectively), as well as diastolic (−1.8 mmHg (SE 0.9) and −2.2 mmHg (SE 1.0), respectively), pressures. However, these reductions were paralleled by similar reductions in the placebo group (systolic −1.7 mmHg (SE 0.9) and diastolic −1.7 mmHg (SE 1.4)) and the pioglitazone group (systolic −3.0 mmHg (SE 0.9) and diastolic −3.9 mmHg (SE 1.5)).

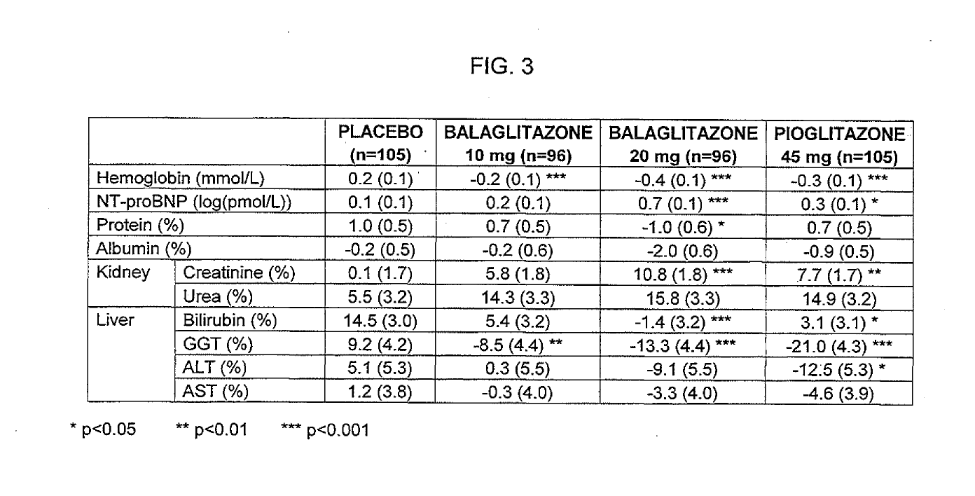

All changes in biochemical safety parameters, such as hemoglobin levels, serum creatinine, serum urea, bilirubin, and ALT/AST/GGT levels were as expected for TZD therapy. The results are shown in FIG. 3, where values in parentheses are standard deviations.

Minor reductions in parameters indicating liver function, hemoglobin levels, and minor increases in parameters of kidney function were observed. Serum levels of N-terminal pro-brain natriuretic peptide (“NT-proBNP”) were monitored and, as seen in FIG. 3, a significant increase in NT-proBNP was seen in both the balaglitazone 20 mg and the pioglitazone 45 mg group, when compared to placebo, while only a trend was observed in the balaglitazone 10 mg group. The increase in the balaglitazone 20 mg was significantly larger than that in the pioglitazone 45 mg group (p<0.01).

Balaglitazone was well-tolerated throughout the study. Similar numbers of subjects reported adverse events in all four treatment groups: 81 out of 99 subjects in the balaglitazone 10 mg group, 77 out of 99 in the balaglitazone 20 mg group, 75 out of 102 in the pioglitazone 45 mg group, and 81 out of 109 in the placebo group. As seen in FIG. 3, the percentage of serious adverse events was low, ranging from 6% in the balaglitazone 10 mg group to 11% in the balaglitazone 20 mg group, and no serious adverse event was over-represented in any treatment group. No subjects died during the study.

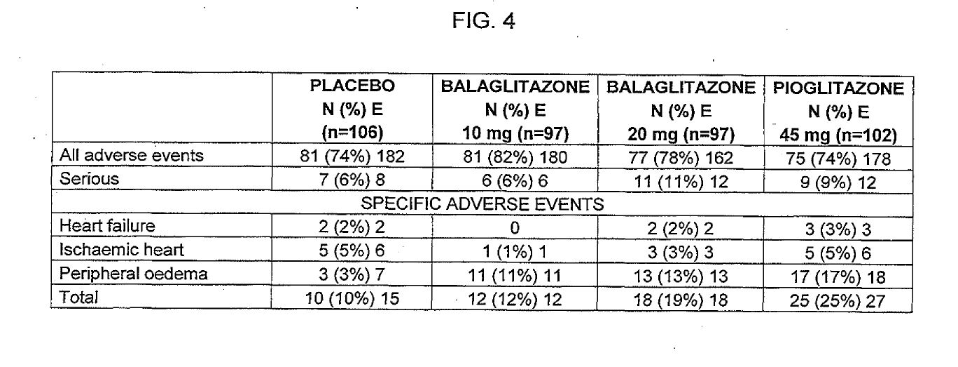

The adverse events (“AEs”) of special interest were all related to strain on the heart or bone fractures, as shown in FIG. 4.

Only one fracture, in the balaglitazone 20 mg group, was observed. The number of AEs of special interest occurred in more subjects receiving pioglitazone 45 mg (25%) and balaglitazone 20 mg (19%), than placebo (12%) or balaglitazone 10 mg (10%). Fisher's Exact Test showed a statistical difference in the frequencies of these AEs between all four groups (p<0.05). Furthermore, when leaving the placebo group out of the analysis, the test still showed significant differences in frequencies between the three treatment groups (p<0.05). However, the study was not powered for individual group comparisons.

Apart from the AEs of special interest, the most frequently reported AE was influenza, which occurred more frequently in pioglitazone 45 mg subjects (9%, total number of events 10) and balaglitazone 20 mg (7%, total number of events 7) subjects than in the balaglitazone 10 mg and placebo groups (3% and 3%, respectively). Furthermore, between 3% (pioglitazone) and 10% (placebo) reported nasopharyngitis.

A total of 16% of the subjects in the balaglitazone 10 mg group withdrew due to an AE, compared with 17% in the balaglitazone 20 mg group, 8% in the pioglitazone 45 mg group, and 15% in the placebo group.

### DISCUSSION OF RESULTS

This study, conducted in a large population of adults with long-standing type 2 diabetes (mean duration 13.5 years), poorly controlled with a stable insulin dose of at least 30 U/day, showed a positive effect of balaglitazone on the primary endpoint, namely a reduction of HbA_{1c}. Effects on reduction of FSG, serum insulin, 7-point glucose tests, and use of insulin were also significant compared with placebo, clearly demonstrating a beneficial profile of balaglitazone on glycemic control in these subjects. With respect to magnitude, the effects of both balaglitazone 10 mg and balaglitazone 20 mg were comparable to the effects seen in the active comparator group (pioglitazone 45 mg).

Because the subjects studied were on stable insulin therapy, insulin usage was monitored, and as seen in previous studies using either rosiglitazone (27) or pioglitazone (28-31) in combination with insulin, an increased probability for decreasing the insulin dose or decreased probability for increasing the dose were observed in all three treatment groups, when compared to placebo. The changes in probability for altering insulin dose were of similar magnitude between the treatment groups. These data confirm that increased insulin sensitivity is obtained with balaglitazone.

With regard to the secondary endpoints, the balaglitazone 10 mg treatment, while leading to increases in both parameters, showed a trend toward lower fluid retention and significantly less fat accumulation than balaglitazone 20 mg and pioglitazone 45 mg, while exerting virtually identical effects on glucose regulation. A previous head-to-head study of pioglitazone 30 mg and 45 mg doses in subjects on stable insulin therapy showed that pioglitazone 45 mg was superior with respect to reduction in glucose parameters, but the weight increases observed were similar in magnitude (2.9 kg versus 3.4 kg, during a 24-week period) (28). Similarly, studies comparing different doses of rosiglitazone and pioglitazone showed a similar incidence of oedema between doses, although differences in glycemic control were observed with both treatments (27, 28). Findings from this study show that balaglitazone, while exerting similar glycemic control to pioglitazone or rosiglitazone, led to less weight gain and less fluid retention (22, 25). These data indicate that the partial mode of action employed by balaglitazone may improve the balance between efficacy and safety in subjects with type-2 diabetes.

Non-fatal heart failure has been shown to be associated with TZD therapy (5, 6, 32, 33), and thus the finding that balaglitazone at the 10 mg dose leads to less fat accumulation, a trend towards reduced fluid retention, and a trend towards fewer heart failures (0 in the balaglitazone 10 mg group, compared to 3 in the pioglitazone group) is promising. In addition, the lipid profile induced by balaglitazone appeared to correlate with the beneficial changes induced by pioglitazone (34), rather than rosiglitazone, for which a similar benefit is not seen on lipid parameters (27).

Total body BMD and serum ALP were monitored in this trial, as TZD therapy has been associated with bone loss due to reduced bone formation rates by osteoblasts, and thus leads to increased fracture rates (5, 15, 17, 35-38). In this study, total body BMD was not statistically altered in any of the treatment groups. However, a trend toward a reduction in BMD was observed in the pioglitazone 45 mg group, while no reductions in BMD were observed in the balaglitazone groups, when compared to placebo. These data were further supported by serum ALP measurements, showing a significantly lower reduction in the balaglitazone 10 mg group than the pioglitazone 45 mg group.

Balaglitazone at both the 10 mg and 20 mg doses produced a satisfactory safety profile. Serious AEs were observed in only a few subjects, and these were equally represented in the pioglitazone and placebo groups as well. For the AEs of special interest, heart failure, peripheral oedema, and myocardial infarction were numerically lower in the balaglitazone 10 mg group than in the remaining groups, indicating that balaglitazone at 10 mg may have a better safety profile with respect to cardiac events. Fisher's Exact Test for frequencies of AEs showed a significant difference in AE frequency between all four groups (p<0.05). A significant difference in AE frequency was also observed between the treatment groups when the placebo group was excluded from the analysis (p<0.05), showing that the placebo group did not drive the difference alone. This indicates that balaglitazone at 10 mg causes fewer adverse events.

Minor changes in blood pressure, biochemical parameters of liver and kidney function, and hemoglobin values were observed in both balaglitazone groups. Although these were correlated to the dose used, they were all well within the normal range of the individual parameters.

The data presented herein illustrates that a 10 mg dose of balaglitazone is equipotent to pioglitazone 45 mg with respect to glucose-lowering capabilities, while causing fewer of the unwanted side effects.

These and other modifications and variations to the present application may be practiced by those of ordinary skill in the art, without departing from the spirit and scope of the present application, which is more particularly set forth in the appended claims. In addition, it should be understood that features of the various embodiments may be interchanged in whole or in part. Furthermore, those of ordinary skill in the art will appreciate that the foregoing description is by way of example only, and is not intended to limit the application so further described in such appended claims. Therefore, the spirit and scope of the appended claims should not be limited to the description of the embodiments contained herein.

### DOCUMENTS

The following documents have been cited in this application, or relate in some manner to treating a condition mentioned herein. The listing of the documents is intended merely to acknowledge the assertions made by their authors, and no admission is made that any document is particularly relevant to the claims.
1. World Health Organization, WHO. Diabetes. Fact sheet no 312. Geneva. 2008.
2. Kopelman P G, Hitman G A. Diabetes. Exploding type II. Lancet 1998 Dec. 19; 352 Suppl 4:SIV5.
3. Geiser A G, Zeng Q Q, Sato M, Helvering L M, Hirano T, Turner C H. Decreased bone mass and bone elasticity in mice lacking the transforming growth factor-betel gene. Bone 1998 August; 23:87-93.

4. Cho N, Momose Y. Peroxisome proliferator-activated receptor gamma agonists as insulin sensitizers: from the discovery to recent progress. Curr Top Med Chem 2008; 8(17):1483-507.
5. Home P D, Pocock S J, Beck-Nielsen H., Curtis P S, Gomis R, Hanefeld M et al. Rosiglitazone evaluated for cardiovascular outcomes in oral agent combination therapy for type 2 diabetes (RECORD): a multicentre, randomised, open-label trial. Lancet 2009 Jun. 20; 373(9681):2125-35.
6. Lincoff A M, Wolski K, Nicholls S J, Nissen S E. Pioglitazone and risk of cardiovascular events in patients with type 2 diabetes mellitus: a meta-analysis of randomized trials. JAMA 2007 Sep. 12; 298(10):1180-8.
7. Nielsen R, Pedersen T A, Hagenbeek D, Moulos P, Siersbaek R, Megens E et al. Genome-wide profiling of PPARgamma:RXR and RNA polymerase II occupancy reveals temporal activation of distinct metabolic pathways and changes in RXR dimer composition during adipogenesis. Genes Dev 2008 Nov. 1; 22(21):2953-67.
8. Nesto R W, Bell D, Bonow R O, Fonseca V, Grundy S M, Horton E S et al. Thiazolidinedione use, fluid retention, and congestive heart failure: a consensus statement from the American Heart Association and American Diabetes Association. Oct. 7, 2003. Circulation 2003 Dec. 9; 108(23):2941-8.
9. Nissen S E, Wolski K. Effect of rosiglitazone on the risk of myocardial infarction and death from cardiovascular causes. N Engl J Med 2007 Jun. 14; 356(24):2457-71.
10. Fonseca V. Effect of thiazolidinediones on body weight in patients with diabetes mellitus. Am J Med 2003 Dec. 8; 115 Suppl 8A:42S-8S.
11. Takada I, Suzawa M, Matsumoto K, Kato S. Suppression of PPAR transactivation switches cell fate of bone marrow stem cells from adipocytes into osteoblasts. Ann N Y Acad Sci 2007 November; 1116:182-95.
12. Karalliedde J, Buckingham R E. Thiazolidinediones and their fluid-related adverse effects: facts, fiction and putative management strategies. Drug Saf 2007; 30(9):741-53.
13. Ali A A, Weinstein R S, Stewart S A, Parfitt A M, Manolagas S C, Jilka R L. Rosigiltazone causes bone loss in mice by suppressing osteoblast differentiation and bone formation. Endocrinology 2005 March; 146(3):1226-35.
14. Rosen C J. The rosiglitazone story—lessons from an FDA Advisory Committee meeting. N Engl J Med 2007 Aug. 30; 357(9):844-6.
15. McDonough A K, Rosenthal R S, Cao X, Saag K G. The effect of thiazolidinediones on BMD and osteoporosis. Nat Clin Pract Endocrinol Metab 2008 September; 4(9):507-13.
16. Rosen C J. Sugar and bone: a not-so sweet story. J Bone Miner Res 2008 December; 23(12):1881-3.
17. Loke Y K, Singh S, Furberg C D. Long-term use of thiazolidinediones and fractures in type 2 diabetes: a meta-analysis. CMAJ 2009 Jan. 6; 180(1):32-9.
18. Lecka-Czernik B, Gubrij I, Moerman E J, Kajkenova O, Lipschitz D A, Manolagas S C et al. Inhibition of Osf2/Cbfa1 expression and terminal osteoblast differentiation by PPARgamma2. J Cell Biochem 1999 Sep. 1; 74(3):357-71.
19. Soroceanu M A, Miao D, Bai X Y, Su H, Goltzman D, Karaplis A C, Rosiglitazone impacts negatively on bone by promoting osteoblast/osteocyte apoptosis. J Endocrinol 2004 October; 183(1):203-16.
20. Berger J P, Petro A E, Macnaul K L, Kelly L J, Zhang B B, Richards K et al. Distinct properties and advantages of a novel peroxisome proliferator-activated protein [gamma] selective modulator. Mol Endocrinol 2003 April; 17(4):662-76.
21. Berger J P, Akiyama T E, Meinke P T. PPARs: therapeutic targets for metabolic disease. Trends Pharmacol Sol 2005 May; 26(5):244-51.
22. Larsen P J, Lykkegaard K, Larsen L K, Fleckner J, Sauerberg P, Wassermann K et al. Dissociation of antihyperglycaemic and adverse effects of partial perioxisome proliferator-activated receptor (PPAR-gamma) agonist balaglitazone. Eur J Pharmacol 2008 Oct. 31; 596(1-3):173-9.
23. Einstein M, Akiyama T E, Castriota G A, Wang C F, McKeever B, Mosley R T et al. The differential interactions of peroxisome proliferator-activated receptor gamma ligands with Tyr473 is a physical basis for their unique biological activities. Mol Pharmacol 2008 January; 73(1):62-74.
24. Burgermeister E, Schnoebelen A, Flament A, Benz J, Stihle M, Gsell B et al. A novel partial agonist of peroxisome proliferator-activated receptor-gamma (PPARgamma) recruits PPARgamma-coactivator-1 alpha, prevents triglyceride accumulation, and potentiates insulin signaling in vitro. MeI Endocrinol 2006 April; 20(4):809-30.
25. Henriksen K, Byrjalsen I, Nielsen R H, Madsen A N, Larsen L K, Christiansen C et al. A comparison of glycemic control, water retention, and musculoskeletal effects of balaglitazone and pioglitazone in diet-induced obese rats. Eur J Pharmacol 2009 Aug. 15; 616(1-3):340-5.
26. Brijker F, Heijdra Y F, Van Den Elshout F J, Bosch F H, Folgering H T. Volumetric measurements of peripheral oedema in clinical conditions. Clin Physiol 2000 January; 20(1):56-61.
27. Raskin P, Rendell M, Riddle M C, Dole J F, Freed M I, Rosenstock J. A randomized trial of rosiglitazone therapy in patients with inadequately controlled insulin-treated type 2 diabetes. Diabetes Care 2001 July; 24(7):1226-32.
28. Davidson J A, Perez A, Zhang J. Addition of pioglitazone to stable insulin therapy in patients with poorly controlled type 2 diabetes: results of a double-blind, multicentre, randomized study. Diabetes Obes Metab 2006 March; 8(2):164-74.
29, Mattoo V, Eckland D, Widel M, Duran S, Fajardo C, Strand J et al. Metabolic effects of pioglitazone in combination with insulin in patients with type 2 diabetes mellitus whose disease is not adequately controlled with insulin therapy: results of a six-month, randomized, double-blind, prospective, multicenter, parallel-group study. Clin Ther 2005 May; 27(5):554-67.
30. Berhanu P, Perez A, Yu S. Effect of pioglitazone in combination with insulin therapy on glycaemic control, insulin dose requirement and lipid profile in patients with type 2 diabetes previously poorly controlled with combination therapy. Diabetes Obes Metab 2007 July; 9(4):512-20.
31. Rosenstock J, Einhorn D, Hershon K, Glazer N B, Yu S. Efficacy and safety of pioglitazone in type 2 diabetes: a randomised, placebo-controlled study in patients receiving stable insulin therapy. Int J Clin Pract 2002 May; 56(4):251-7.
32. Erdmann E, Charbonnel B, Wilcox R G, Skene A M, Massi-Benedetti M, Yates J et al. Pioglitazone use and heart failure in patients with type 2 diabetes and preexisting cardiovascular disease: data from the PROactive study (PROactive 08). Diabetes Care 2007 November; 30(11):2773-8.
33. Singh S, Loke Y K, Furberg C D. Long-term risk of cardiovascular events with rosiglitazone: a meta-analysis. JAMA 2007 Sep. 12; 298(10):1189-95.
34. Henry R R, Lincoff A M, Mudaliar S, Rabbia M, Chognot C, Herz M. Effect of the dual peroxisome proliferator-activated receptor-alpha/gamma agonist aleglitazar on risk of cardiovascular disease in patients with type 2 diabetes (SYNCHRONY): a phase II, randomised, dose-ranging study. Lancet 2009 Jul. 11; 374(9684):126-35.
35. Schwartz A V. TZDs and Bone: A Review of the Recent Clinical Evidence. PPAR Res 2008; 2008:297893.
36. Grey A, Bolland M, Gamble G, Wattle D, Horne A, Davidson J et al. The peroxisome proliferator-activated receptor-gamma agonist rosiglitazone decreases bone formation and bone mineral density in healthy postmenopausal women: a randomized, controlled trial. J Clin Endocrinol Metab 2007 April; 92(4):1305-10.
37. Dormuth C R, Carney G, Carleton B, Bassett K, Wright J M. Thiazolidinediones and fractures in men and women. Arch Intern Med 2009 Aug. 10; 169(15):1395-402.
38. Douglas I J, Evans S J, Pocock S, Smeeth L. The risk of fractures associated with thiazolidinediones: a self-controlled case-series study. PLoS Med 2009 September; 6(9):e1000154.
39. Gaede P, Hildebrandt P, Hess G, Parving H H, Pedersen O. Plasma N-terminal pro-brain natriuretic peptide as a major risk marker for cardiovascular disease in patients with type 2 diabetes and microalbuminuria. Diabetologia 2005 January; 48(1):156-63.
40. Chang A M, Maisel A S, Hollander J E. Diagnosis of heart failure. Heart Fail Clin 2009 January; 5(1):25-35, vi.
41. Dorkhan M, Dencker M, Stagmo M, Groop L. Effect of pioglitazone versus insulin glargine on cardiac size, function, and measures of fluid retention in patients with type 2 diabetes. Cardiovasc Diabetol 2009; 8:15.

1.-40. (canceled)

41. A method for improving or maintaining bone density or bone mineral content, or both, in a subject suffering from insulin resistance comprising administering balaglitazone in an amount effective to improve or maintain bone density while decreasing insulin resistance.

42. A method of reducing the risk or incidence of bone fracture in a subject suffering from insulin resistance comprising administering balaglitazone in an amount effective to reduce the risk or incidence of bone fracture while decreasing insulin resistance.

43. A method of decreasing the rate of bone degradation in a subject suffering from insulin resistance comprising administering balaglitazone in an amount effective to decrease the rate of bone degradation while decreasing insulin resistance.

44. A method of reducing bone loss in a subject suffering from insulin resistance comprising administering balaglitazone in an amount effective to decrease reduce the bone loss while decreasing insulin resistance.

45. The method of claim 41, 42, 43, or 44, wherein the subject is a human.

46. The method of claim 41, 42, 43, or 44, wherein a rate of bone formation by osteoblasts is maintained during said administration.

47. The method of claim 41, 42, 43, or 44, wherein blood glucose control is improved in the subject.

48. The method of claim 41, 42, 43, or 44, wherein said subject is selected from population of patients suffering from an increased risk of bone fracture related to the patients' population's age, sex, and weight demographics.

49. The method of claim 41, 42, 43, or 44, wherein the subject is an adult and balaglitazone is administered to the subject at a dose from about 5 mg to about 15 mg per day.

50. The method of claim 49, wherein the dose is from about 9 to about 12 mg per day.

51. The method of claim 49, wherein the dose is from about 9.5 to about 10.5 mg per day.

52. The method of claim 49, wherein the dose is about 5 mg per day.

53. The method of claim 41, 42, 43, or 44, further comprising administering an effective amount of insulin to said subject.

54. The method of claim 50, wherein the subject receives stable insulin therapy.

55. The method of claim 41, 42, 43, or 44, wherein the subject has a condition selected from the group consisting one or more of type 2 diabetes, dyslipidemia, hyperglycemia, hyperinsulinemia, insulin resistance, obesity, cardiovascular complications, atherosclerosis, hypertension, impaired glucose tolerance, impaired fasting glucose level, increased plasma levels of free fatty acids, increased plasma levels of triglycerides, and increased plasma levels of very low density lipoproteins.

56. The method of claim 41, 42, 43, or 44, wherein the subject has a condition including one or more of osteoporosis, achondroplasia, bone and joint infections, bone cancer, bone fractures, craniosynostosis, curvature of the spine, enchondroma, fibrodysplasiaossificans progressive, fibrous dysplasia, giant cell tumor of bone, infectious arthritis, Klippel-feil syndrome, Legg Calve Perthes disease, myeloma, Osgood-Schlater disease, OsteitisCondensansllii, osteochondritisdissecans-OCD, osteochondroma—bone tumor, osteogenesis, osteomalacia, osteomyelitis, osteonecrosis, osteopenia, osteopetrosis, osteophytes, Paget's disease, Proteus syndrome, renal osteodystrophy, rickets, scoliosis, charcot joint, diabetic hand syndrome, osteoarthritis, diffuse idiopathic skeletal hyperostosis, Dupuytren contracture, frozen shoulder, and unicameral bone cyst.

57. The method of claim 41, 42, 43, or 44, wherein a serum bone alkaline phosphatase level of the subject after balaglitazone administration is substantially maintained at a pre-administration level.

58. The method of claim 41, 42, 43, or 44, wherein a serum osteocalcin level of the subject after balaglitazone administration is substantially maintained at a pre-administration level.

59. The method of claim 41, 42, 43, or 44, wherein a bone mineral density of the subject after balaglitazone administration is substantially maintained at a pre-administration level.

60. The method of claim 41, 42, 43, or 44, further comprising administering an additional therapeutic agent for restricting bone loss.

61. The method of claim 60, wherein the additional therapeutic agent for restricting bone loss is selected from the group consisting of one or more of vitamin A, vitamin D2, vitamin D, calcium, an analgesic, a non-steroidal anti-inflammatory drug, a cyclooxygenase-2 inhibitor, acetaminophen, a chemotherapy drug, an antibiotic, an antifungal, a corticosteroid, and cinacalcet hydrochloride.

62. A method for providing blood glucose control in a subject suffering from a condition benefiting from a decrease in insulin resistance, wherein the subject is selected based upon an increased risk of bone fracture for the subject's age, sex, and/or weight demographics, the method comprising administering balaglitazone to the subject.

63. The method of claim 41, 42, 43, or 44, comprising decreasing insulin resistance in said subject, wherein the subject has a condition selected from the group consisting one or more of type 2 diabetes, dyslipidemia, hyperglycemia, hyperinsulinemia, insulin resistance, obesity, cardiovascular complications, atherosclerosis, hypertension, impaired glucose tolerance, impaired fasting glucose level, increased plasma levels of free fatty acids, increased plasma levels of triglycerides, and increased plasma levels of very low density lipoproteins.
